(19) United States
(12) Patent Application Publication
(10) Pub. No.: US 2026/0198763 A1
(43) Pub. Date: Jul. 16, 2026

(54) INTRALUMINAL PRESSURE LIMITER EFFICACY ASSESSMENT SYSTEM

(71) Applicant: BOSTON SCIENTIFIC SCIMED, INC., Maple Grove, MN (US)

(72) Inventors: Jessica Williams, Marlborough, MA (US); Alycia Abbott, Groton, MA (US); Candace A. Rhodes, Hopkinton, MA (US); Aditi Ray, Palo Alto, CA (US)

(73) Assignee: BOSTON SCIENTIFIC SCIMED, INC., Maple Grove, MN (US)

(21) Appl. No.: 19/445,155

(22) Filed: Jan. 9, 2026

Related U.S. Application Data
(60) Provisional application No. 63/744,009, filed on Jan. 10, 2025.

(51) Int. Cl. A61B 1/015; A61B 1/00; A61B 1/12; A61B 1/307

(52) U.S. Cl. CPC A61B 1/015; A61B 1/00006; A61B 1/0004; A61B 1/00055; A61B 1/128; A61B 1/307; A61B 2562/0247

(57) ABSTRACT

A system for monitoring intraluminal pressure during endoscopic procedures may include a fluid management system configured to deliver fluid to patient anatomy and an endoscope configured to measure intraluminal pressure. A controller may be configured to calculates an area under the curve metric between a maximum pressure value and measured intraluminal pressure over time when the measured pressure exceeds the maximum value. The controller may compare the calculated metric to a predetermined area under the curve value and reduce fluid flow if the calculated metric exceeds the predetermined value.

### CROSS-REFERENCE TO RELATED APPLICATIONS

The application claims the benefit of U.S. Provisional Patent Application Ser. No. 63/744,009, filed on Jan. 10, 2025, the disclosure of which is incorporated herein by reference.

### TECHNICAL FIELD

The disclosure is directed to a fluid management system. More particularly, the disclosure is directed to methods and systems for flow control in a fluid management system.

### BACKGROUND

Flexible ureteroscopy (fURS), gynecology, and other endoscopic procedures require the circulation of fluid for several reasons. Surgeons today deliver the fluid in various ways such as, for example, by hanging a fluid bag and using gravity to deliver the fluid, filling a syringe and manually injecting the fluid or using a peristaltic pump to deliver fluid from a reservoir at a fixed pressure or flowrate via a fluid management system. Fluid management systems may adjust the flowrate and/or pressure at which fluid is delivered from the reservoir based on data collected from a procedural device, such as, but not limited to, an endoscope. These systems incorporate pressure monitoring capabilities to measure intraluminal pressure (ILP) within anatomical structures during procedures. Advanced fluid management systems employ automated control mechanisms that regulate fluid flow based on real-time pressure measurements. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In an example, a system for monitoring intraluminal pressure during endoscopic procedures may include a fluid management system configured to deliver fluid to a patient anatomy, an endoscope configured to measure intraluminal pressure, and a controller. The controller may be configured to calculate an area under the curve metric between a maximum pressure and a measured intraluminal pressure over time when the measured intraluminal pressure exceeds the maximum value, compare the calculated area under the curve metric to a predetermined area under the curve value, and if the calculated area under the curve metric is greater than the predetermined area under the curve value, reduce a flow of fluid from the fluid management system.

Alternatively or additionally to any of the examples above, in another example, calculating the area under the curve metric may include integrating the difference between the measured intraluminal pressure and the maximum pressure from a time when the intraluminal pressure exceeds the maximum pressure to a time when the intraluminal pressure returns below the maximum pressure.

Alternatively or additionally to any of the examples above, in another example, calculating the area under the curve metric may include sampling the intraluminal pressure values at discrete time points and performing a numerical approximation using the sampled intraluminal values.

Alternatively or additionally to any of the examples above, in another example, the maximum pressure may be determined by a mode of operation of the endoscope.

Alternatively or additionally to any of the examples above, in another example, a maximum pressure for a flush mode may be greater than a maximum pressure for a base flow operation.

Alternatively or additionally to any of the examples above, in another example, if the calculated area under the curve metric exceeds the predetermined area under the curve value, the controller may be configured to reduce a speed of an inflow pump delivering the fluid.

Alternatively or additionally to any of the examples above, in another example, the predetermined area under the curve value may be between 130-150 mmHg·seconds for base flow operations and between 250-280 mmHg·seconds for flush operations.

Alternatively or additionally to any of the examples above, in another example, the controller may be further configured to calculate a cumulative area under the curve metric for multiple pressure overshoots during a procedure and compare the cumulative area under the curve metric to a predetermined cumulative value.

Alternatively or additionally to any of the examples above, in another example, the controller may be configured to generate an alert when the calculated area under the curve metric exceeds the predetermined area under the curve value.

Alternatively or additionally to any of the examples above, in another example, the controller may be configured to increase fluid flow at a reduced rate after the measured intraluminal pressure returns below the maximum pressure following an overshoot event.

In an example, a non-transitory computer-readable medium storing instructions that, when executed by a processor, may cause the processor to receive intraluminal pressure measurements from a pressure sensor during a procedure, detect when a measured intraluminal pressure exceeds a maximum pressure value, calculate an area under the curve metric between the maximum pressure value and the measured intraluminal pressure over time when the measured intraluminal pressure exceeds the maximum value, compare the calculated area under the curve metric to a predetermined area under the curve value, and if the calculated area under the curve metric exceeds the predetermined area under the curve value, generate a control signal to reduce fluid flow.

Alternatively or additionally to any of the examples above, in another example, calculating the area under the curve metric may include sampling the intraluminal pressure measurements at discrete time points and performing a trapezoidal numerical approximation using the intraluminal pressure measurements.

Alternatively or additionally to any of the examples above, in another example, the predetermined area under the curve value may be between 130-150 mmHg·seconds for base flow operations and between 250-280 mmHg·seconds for flush operations.

Alternatively or additionally to any of the examples above, in another example, the instructions may further cause the processor to calculate a cumulative area under the curve metric for multiple pressure overshoots during the procedure and compare the cumulative area under the curve metric to a predetermined cumulative specification.

Alternatively or additionally to any of the examples above, in another example, the instructions may further cause the processor to generate an alert when the calculated area under the curve metric exceeds the predetermined area under the curve value.

In an example, a system for monitoring intraluminal pressure during endoscopic procedures may include a fluid management system configured to deliver fluid to a patient anatomy, an endoscope configured to measure intraluminal pressure, and a controller configured to calculate an area under the curve metric between a maximum pressure and a measured intraluminal pressure over time when the measured intraluminal pressure exceeds the maximum value, compare the calculated area under the curve metric to a predetermined area under the curve value, and if the calculated area under the curve metric is greater than the predetermined area under the curve value, reduce a flow of fluid from the fluid management system.

Alternatively or additionally to any of the examples above, in another example, calculating the area under the curve metric may include integrating the difference between the measured intraluminal pressure and the maximum pressure from a time when the intraluminal pressure exceeds the maximum pressure to a time when the intraluminal pressure returns below the maximum pressure.

Alternatively or additionally to any of the examples above, in another example, calculating the area under the curve metric may include sampling the intraluminal pressure values at discrete time points and performing a numerical approximation using the sampled intraluminal values.

Alternatively or additionally to any of the examples above, in another example, the maximum pressure may be determined by a mode of operation of the endoscope.

Alternatively or additionally to any of the examples above, in another example, a maximum pressure for a flush mode may be greater than a maximum pressure for a base flow operation.

Alternatively or additionally to any of the examples above, in another example, if the calculated area under the curve metric exceeds the predetermined area under the curve value, the controller may be configured to reduce a speed of an inflow pump delivering the fluid.

Alternatively or additionally to any of the examples above, in another example, the predetermined area under the curve value may be between 130-150 mmHg·seconds for base flow operations and between 250-280 mmHg·seconds for flush operations.

Alternatively or additionally to any of the examples above, in another example, the controller may be further configured to calculate a cumulative area under the curve metric for multiple pressure overshoots during a procedure and compare the cumulative area under the curve metric to a predetermined cumulative value.

Alternatively or additionally to any of the examples above, in another example, the controller may be configured to generate an alert when the calculated area under the curve metric exceeds the predetermined area under the curve value.

Alternatively or additionally to any of the examples above, in another example, the controller may be configured to increase fluid flow at a reduced rate after the measured intraluminal pressure returns below the maximum pressure following an overshoot event.

In an example, a non-transitory computer-readable medium storing instructions that, when executed by a processor, may cause the processor to receive intraluminal pressure measurements from a pressure sensor during a procedure, detect when a measured intraluminal pressure exceeds a maximum pressure value, calculate an area under the curve metric between the maximum pressure value and the measured intraluminal pressure over time when the measured intraluminal pressure exceeds the maximum value, compare the calculated area under the curve metric to a predetermined area under the curve value, and if the calculated area under the curve metric exceeds the predetermined area under the curve value, generate a control signal to reduce fluid flow.

Alternatively or additionally to any of the examples above, in another example, calculating the area under the curve metric may include sampling the intraluminal pressure measurements at discrete time points and performing a trapezoidal numerical approximation using the intraluminal pressure measurements.

Alternatively or additionally to any of the examples above, in another example, the predetermined area under the curve value may be between 130-150 mmHg·seconds for base flow operations and between 250-280 mmHg·seconds for flush operations.

Alternatively or additionally to any of the examples above, in another example, the instructions may further cause the processor to calculate a cumulative area under the curve metric for multiple pressure overshoots during the procedure and compare the cumulative area under the curve metric to a predetermined cumulative specification.

Alternatively or additionally to any of the examples above, in another example, the instructions may further cause the processor to generate an alert when the calculated area under the curve metric exceeds the predetermined area under the curve value.

In an example, a method of evaluating intraluminal pressure safety during a procedure may include receiving intraluminal pressure measurements from a pressure sensor during fluid delivery to a patient anatomy, detecting when measured intraluminal pressure exceeds a maximum pressure value, calculating an area under the curve metric between the maximum pressure value and the measured intraluminal pressure over time when the measured intraluminal pressure exceeds the maximum value, comparing the calculated area under the curve metric to a predetermined area under the curve value, and if the calculated area under the curve metric exceeds the predetermined area under the curve value, reducing a flow rate of fluid delivery.

Alternatively or additionally to any of the examples above, in another example, calculating the area under the curve metric may include sampling the intraluminal pressure measurements at discrete time points and performing a trapezoidal numerical approximation using the intraluminal pressure measurements.

Alternatively or additionally to any of the examples above, in another example, the predetermined area under the curve value may be between 130-150 mmHg·seconds for base flow operations and between 250-280 mmHg·seconds for flush operations.

Alternatively or additionally to any of the examples above, in another example, the method may further include calculating a cumulative area under the curve metric for multiple pressure overshoots during the procedure and comparing the cumulative area under the curve metric to a predetermined cumulative specification.

Alternatively or additionally to any of the examples above, in another example, the method may further include, after reducing the flow rate of fluid delivery, increasing the flow rate at a reduced rate when the measured intraluminal pressure returns below the maximum pressure value. The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a schematic view of an illustrative medical device that may be used in conjunction with the fluid management system of FIGS. 1-2;

FIG. 4 is an illustrative graph of intraluminal pressure over time during a portion of a procedure; and

FIG. 5 is another illustrative graph of intraluminal pressure over time during a portion of a procedure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ Elite endoscope device using pressure and/or temperature data from the endoscope or other endoscopic device. While the present disclosure is described with respect to urological procedures, the systems and methods described herein may be used in other anatomies, as desired. Direct regulation of the intraluminal pressure during a medical procedure may allow the fluid management system to safely drive pump pressures of up to 600 millimeters of mercury (mmHg) to ensure no loss of flow during the procedure when tools are inserted into the working channel of the endoscope device. Irrigation flowrate and intraluminal pressure (ILP) may affect the efficacy and safety of many urological procedures.

Some fluid management systems may include a flow limiter safety feature which stops the fluid pump if the ILP exceeds the set limiter threshold (e.g., exceeds a predetermined maximum allowable pressure). However, after disabling the pump, fluid can continue to flow from the system to the kidney (or other anatomy). Depending on the volume of outflow from the kidney, the ILP can continue to rise and overshoot the limiter threshold. In some cases, the flow limiter may aim to minimize overshoots, without fully compromising performance. However, due to compliance (e.g., flexibility) in the anatomy, the fluid management system cassette, and the patient line, overshoots may occur even with a flow limiter safety feature. Thus, it may be necessary and/or desirable to define what excursions above the limiter threshold may be acceptable for the pressure limiter feature to still be deemed effective. For example, using either an allowed magnitude (e.g., pressure) or an allowed duration above the limit will only give partial information about the nature of the overshoot. Rather, a combination of both high ILP magnitude, as well as prolonged duration spent at elevated ILPs, may be correlated with absorption volume, and therefore may lead to adverse patient outcomes, such as, but not limited to, sepsis. This disclosure is directed towards methods and systems for monitoring a clinically relevant metric for defining the safety and efficacy of the flow limiter feature.

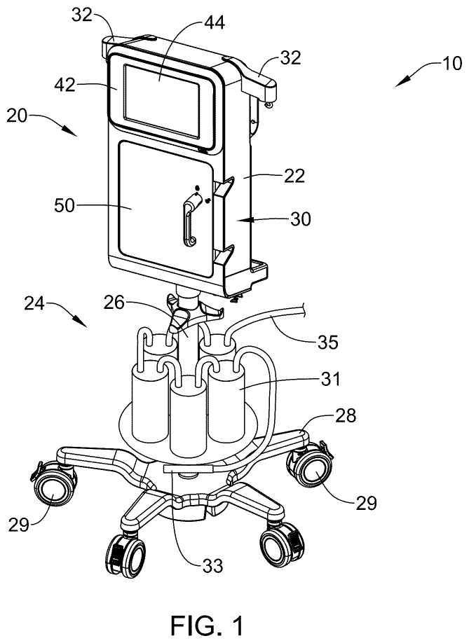

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the Litho Vue™ Elite endoscope, or other endoscope. In some instances, the endoscope may include a temperature sensor to provide intraluminal temperature feedback to the fluid management system 10, a pressure sensor to provide intraluminal pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28 including a plurality of wheels 29 (e.g., caster wheels). In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms, information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined thresholds and/or ranges. In some embodiments, the fluid management system 10 may also include further user interface components such as an optional foot pedal, a fluid warmer user interface, a fluid control interface, or other devices to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, an outflow or vacuum pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The controller 30 may take many forms, including, for example, a microcontroller or microprocessor, coupled to a memory storing readable instructions for performing methods as described herein, as well as providing configuration of the controller 30 for the various examples that follow. The controller 30 may include one more application-specific integrated circuits (ASIC) to provide additional or specialized functionality, such as, without limitation a signal processing ASIC that can filter received signals from one or more sensors using digital filtering techniques. Logic circuitry, state machines, and discrete or integrated circuit components may be included as well. The skilled person will recognize many different hardware implementations are available for a controller.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate or the fluid pressure based on previously set parameters.

An illustrative fluid management unit may include one or more fluid container supports, such as fluid supply source hangers 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers 31, for collecting waste fluid during a medical procedure. The collection containers 31 (e.g., canisters) may be in fluid communication with a vacuum or outflow pump 33 to provide suction for drawing fluid into the collection containers 31. The vacuum pump 33 may be operatively and/or electronically connected to the controller 30. In some embodiments, the vacuum pump 33 may be disposed within the fluid management system 10. Other configurations are also contemplated. In some embodiments, the collection container(s) 31 may be operatively coupled to a collection load cell to detect placement and/or weight of fluid in the collection container(s) to contribute to a fluid deficit calculation. The collection container(s) 31 may be fluidly connected to the medical device via a flexible aspiration tubing 35. The aspiration tubing 35 may be fluidly connected to the medical device in a number of different configurations. For example, the aspiration tubing 35 may be fluidly connected to an aspiration port on the medical device, to an access sheath disposed over the medical device, to a tool configured to be inserted into the working channel of the medical device, etc.

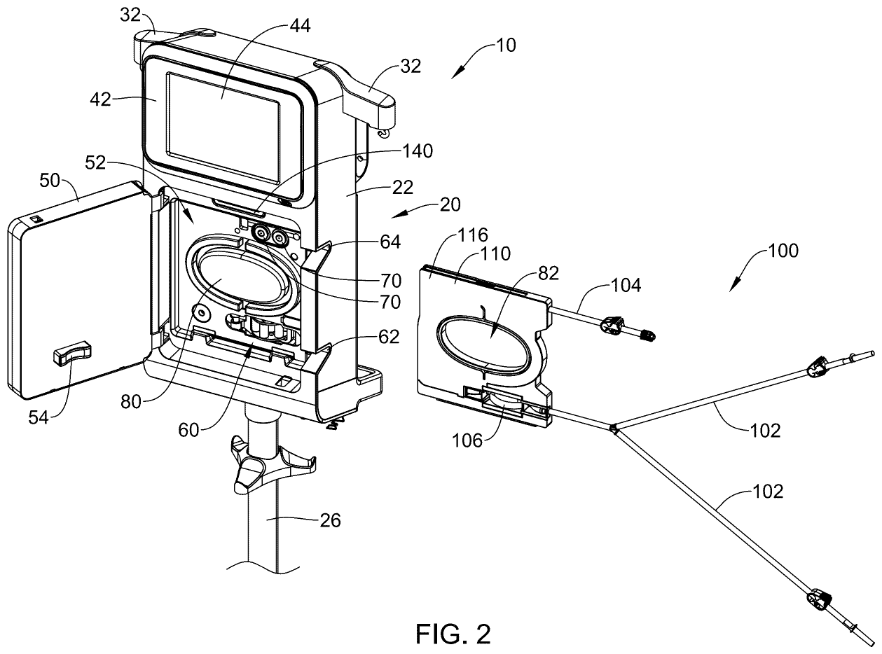

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. The inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target or predetermined fluid flowrate or target fluid pressure based on a set of system operating parameters. In some embodiments, the controller 30 may be configured to control the inflow pump 60 to maintain a desired fluid pressure at the treatment site or a predetermined flowrate based on a set of system operating parameters. In some configurations, the controller 30 may include a flow limiter safety feature. For example, the controller 30 may be configured to control the inflow pump 60 based on, or at least partially on, a limiter threshold (e.g., a maximum allowed intraluminal pressure). The controller 30 may include limiter thresholds for base flow and a limiter threshold for flush operations. The limiter threshold for flush operations may be at least equal to or greater than the limiter threshold for base flow.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may then adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector, for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

The console 20 may further include one or more additional sensors, such as a pressure sensor and/or a bubble sensor. For instance, the console 20 may include a pressure sensor 70, illustrated as a pair of pressure sensors, configured to monitor a system pressure (i.e., pump pressure) of fluid exiting the cassette 110 and flowing through the outflow tubing 104 to a surgical site. The fluid cassette 110 may include a corresponding pressure sensor interface (not explicitly shown), such as a flexible membrane, that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44. Additional features of the cassette 110 of the fluid tubing set 100 are described in commonly assigned U.S. Patent Application No. 63/640,089, titled DEVICES, SYSTEMS, AND METHODS FOR FLOW COMPENSATION IN A FLUID MANAGEMENT SYSTEM, the entire disclosure of which is hereby incorporated by reference.

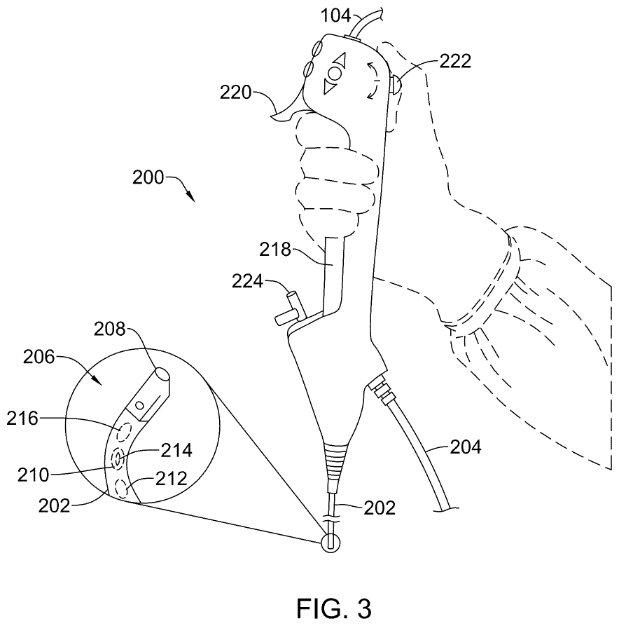

FIG. 3 illustrates aspects of a medical device 200 that may be used in conjunction with the fluid management system 10. In the illustrated embodiments, the medical device 200 may be a ureteroscope such as a LithoVue™ Elite endoscope, another intraluminal pressure sensing endoscope, or other endoscope. However, other medical devices, such as another endoscope, may be used in addition to or in place of a ureteroscope. The medical device 200 may be configured to deliver fluid from the fluid management system 10 to the treatment site via an elongate shaft 202 configured to access the treatment site within the patient. In some embodiments, the inflow pump 60 may be in fluid communication with the elongate shaft 202. The elongate shaft 202 may include one or more working lumens for receiving a flow of fluid or other medical devices therethrough. The medical device 200 is connected to the fluid management system 10 via one or more supply line(s) 104 (e.g., a tube), as shown in FIG. 2 for example.

In some embodiments, the medical device 200 may be in electronic communication with a workstation (not explicitly shown) via a wired connection 204. The workstation may be in wired or wireless communication with the controller 30 of the fluid management system 10. In some embodiments, the workstation may be a multi-use component (e.g., used for more than one procedure) while the medical device 200 may be a single use device, although this is not required. In some embodiments, the workstation may be omitted and the medical device 200 may be electronically coupled directly to the controller 30 of the fluid management system 10.

As shown in FIG. 3, the medical device 200 may include one or more sensors proximate a distal end 206 of the elongate shaft 202. For example, the medical device 200 may include a pressure sensor 208 at a distal tip of the elongate shaft 202 to measure intraluminal pressure within the treatment site. The medical device 200 may also include other sensors such as, for example, a temperature sensor 210, a Fiber Bragg grating optical fiber 212 to detect stresses, and/or an antenna or electromagnetic sensor 214 (e.g., a position sensor). In an illustrative embodiment, the distal end 206 of the medical device 200 may also include at least one camera 216 to provide a visual feed to the user on the display screen of the workstation. In another embodiment, the medical device 200 may include two cameras 216 having different communications requirements or protocols so that different information may be relayed to the user by each camera 216. When so provided, the user may switch back and forth between cameras 216 at will through the touch screen interface 42 and/or the workstation. While not explicitly shown, the elongate shaft 202 may include one or more working lumens for receiving the fluid and/or other medical devices.

The medical device 200 includes a handle 218 coupled to a proximal end of the elongate shaft 202. The handle 218 may have a fluid flow on/off switch 220, which allows the user to control when fluid is flowing through the medical device 200 and into the treatment site. The handle 218 may further include other buttons 222 that perform other various functions. For example, in some embodiments, the handle 218 may include buttons to control the temperature of the fluid. It will be understood that while the exemplary embodiment describes a ureteroscope, the features detailed above may also be directly integrated into a cystoscope, an endoscope, a hysteroscope, or virtually any device with an image capability. In some embodiments, the medical device 200 may also include a drainage port 224 which may be connected to a drainage system such as the vacuum pump 33 and the collection containers 31. Some illustrative drainage systems are described in commonly assigned U.S. Patent Application Publication No. 2018/0361055, titled AUTOMATED FLUID MANAGEMENT SYSTEM, the disclosure of which is hereby incorporated by reference.

The inflow pump 60 and the outflow pump 33 may be controlled to allow the clinician to directly set a flowrate and/or an intraluminal pressure (ILP) for a particular procedure. The fluid management system 10 may then be controlled to maintain the desired flowrate and/or ILP. For example, the controller 30 may be configured to control a pump speed of both the inflow pump 60 and the outflow pump 33 to achieve a desired flowrate and/or ILP. Further, the controller 30 may be configured to slow or stop the inflow pump 60 in response to the measured ILP exceeding a predetermined maximum threshold. The controller 30 may be configured to monitor both the pressure differential between the ILP and the maximum threshold pressure as well as a length of time the ILP exceeds the maximum threshold pressure. For example, the controller 30 may be configured to integrate the ILP over time to determine when to adjust the pump speed of the inflow pump 60 and/or outflow pump 33 to control the ILP. It is contemplated that using only a magnitude of the pressure overshoot or a duration of the overshoot (without taking into consideration magnitude) may cause the controller 30 to adjust the flow rate for clinically insignificant deviations from the maximum ILP. For example, if the fluid management system 10 was configured to adjust inflow pump 60 and/or the outflow pump 33 in response to an ILP of 15 mmHg over the maximum base flow ILP (limiter threshold) of any duration, an overshoot of 16 mmHg for one second would fail the requirement. Similarly, if the fluid management system 10 was configured to adjust inflow pump 60 and/or the outflow pump 33 in response to an overshoot of any magnitude that occurs for a duration of five second or more, an overshoot of 1 mmHg for six seconds would fail the requirement. However, each of these examples are likely to be clinically insignificant due to their short duration and low magnitude, respectively. Integrating the ILP over time may allow the controller 30 to operate the inflow pump 60 and/or the outflow pump 33 in a manner which safely delivers fluid to the patient without compromising performance of the endoscope 200 or clinician visibility.

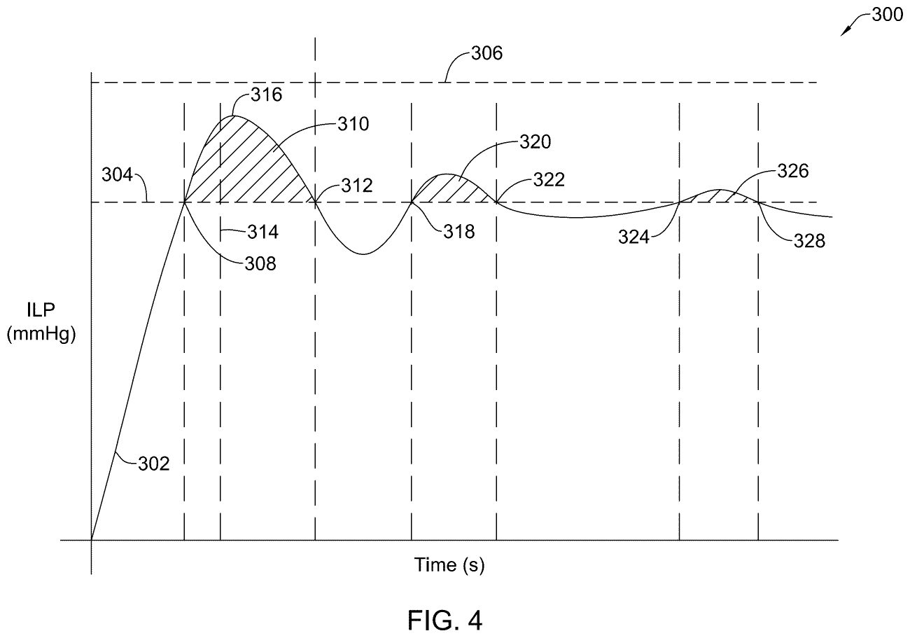

FIG. 4 is an illustrative graph 300 of intraluminal pressure 302 over time during a portion of a procedure. The intraluminal pressure 302 is transmitted from a pressure sensor 208 on the endoscope 200 to the controller 30. The fluid management system 10 may include flow limiter control settings which include a maximum ILP 304 for base flow operations or mode (or normal operation) and a maximum ILP 306 for flush mode or flush operation. As a greater volume of fluid may be introduced through the endoscope 200 for an expected shorter length of time the maximum ILP 306 for the flush mode may be greater than the maximum ILP 304 for flow mode. However, this is not required. The ILP may be measured in mmHg while time may be measured in seconds(s). The maximum ILP 304, 306 may be selected by the user at the touch screen interface 42 at the start of a procedure. For example, the user may select a maximum ILP 304, 306 between a range of about 5 mmHg to 300 mmHg. The maximum ILP 304 for base flow operations and the maximum ILP 306 for flush mode may be selected separately. However, the maximum ILP 306 for flush mode may be required to be at least equal to or greater than the maximum ILP 304 for base flow operations. When the flush mode is activated (e.g., via a button on the endoscope 200), the maximum ILP 306 for flush mode overrides the maximum ILP 304 for base flow operations.

At the beginning of a procedure, the endoscope 200 may be inserted into a body lumen of the patient. When the distal end 206 of the endoscope 200 is at or near the target location, a flow of fluid may be provided from the fluid management system 10 to the endoscope 200. As fluid is provided to the body lumen, the ILP 302 may gradually increase as fluid accumulates in the body lumen. In some cases, at some point during the procedure, the ILP 302 may exceed the maximum ILP 304. In the example illustrated in FIG. 4, the ILP 302 initially exceeds the maximum ILP 304 for flow operations at a first time 308. Once the ILP 302 exceeds the maximum ILP 304 for flow operations, the controller 30 may begin to integrate the ILP over time to determine the “area under the curve” illustrated as shaded region 310 between the measured ILP 302 and the maximum ILP 304 for flow operations. This may be given as the integral equation:

$$AUC = {\int }_{time measured ILP exceeds reference}^{time measured ILP drops below reference} ( ILP - Reference ) dt \qquad Equation 1$$

where AUC equals the area under the curve, ILP is the measure intraluminal pressure, and reference is the maximum ILP set by the user, a baseline pressure of the patient, or atmospheric pressure. The maximum ILP may vary depending on the mode of operation of the fluid management system 10. For example, as noted above, when the fluid management system 10 is in a flush mode, the maximum ILP may be greater than when the fluid management system 10 is in base flow mode. The maximum ILP will be used as the reference pressure for the description of calculating area under the curve and control of the fluid flow. It should be understood that the baseline pressure of the patient or atmospheric pressure may be substituted for a user defined maximum pressure, as desired. The area under the curve may be a metric used to determine if the magnitude of pressure overshoot and duration of the pressure overshoot may cause harm to the patient. Further, the area under the curve may be a metric used to determine when to adjust one or more parameters of the fluid management system 10, such as, but not limited to, a speed of the inflow pump 60, a direction of the inflow pump 60, a speed of the outflow pump 33, or the like, to maintain patient safety without sacrificing performance.

As the controller 30 is receiving the measured ILP 302 as discrete measurements at predetermined time intervals, the controller 30 may be configured to use numerical approximation to determine the area between each ILP measurement which are added together. Said differently, the controller 30 may use a trapezoidal approximation method. This may break the area under the curve 310 into small trapezoids using sequential pressure measurements and sum the incrementally obtained areas. It is contemplated that the controller 30 may be configured to cumulatively sum the area under the curve 310 as new pressure measurements are received. The controller 30 may be configured to continue to sum the area under the curve 310 until the ILP 302 drops below the maximum pressure 304 for base flow operations, as shown at time 312. Further, the controller 30 may be configured to compare each summation (e.g., the summation after each new pressure measurement is received) to a predetermined area under the curve value for an event. An event may be a single excursion above the maximum pressure 304 for base operations (or maximum pressure 306 for flush mode) from the time ILP 302 first exceeds the maximum pressure to the time the ILP 302 drops below the maximum pressure. For example, FIG. 4 illustrates three pressure events. The predetermined area under the curve value for a pressure event may be stored in a memory of the controller 30. While the calculation of the area under the curve is described with respect to the ILP 302 exceeding the maximum pressure 304 for base operations, the controller 30 is also configured to calculate the area under the curve when flush mode is active, using the maximum pressure 306 for flush mode as the trigger to start and stop calculating the area under the curve.

If at any time between the time 308 the ILP 302 exceeds the maximum pressure 304 for base flow operations and the time 312 the ILP 302 drops below the maximum pressure 304 for base flow operations the area under the curve 310 exceeds the predetermined area under the curve value (having units of pressure multiplied by time (e.g., mmHg·second)), the controller 30 may be configured to control a speed of the inflow pump 60, a direction of the inflow pump 60 (e.g., the pump 60 may be reversed to draw fluid out), and/or a speed of the outflow pump 33 to lower the intraluminal pressure. For example, the area under the curve 310 may exceed the predetermined area under the curve value at an intermediate time 314 between the first time 308 and the second time 312. The controller 30 may then transmit a control signal to the inflow pump 60 to reduce the pump speed to deliver less fluid to lower the ILP 302 without user intervention. The ILP 302 may continue to rise for a short period of time after the controller 30 reduces the pump speed. This may be due to compliance (e.g., flexibility) in the components of the fluid management system 10, the patient, etc. After a peak 316, the ILP 302 may begin to drop. Once the ILP 302 is less than the maximum pressure 304 for base flow operations, the controller 30 may stop calculating the area under curve. In some examples, the controller 30 may store the area under curve 310 to use for analysis (e.g., future modifications of acceptable area under the curve values) and/or to determine a cumulative area under the curve for an entire procedure. Additionally, once the ILP 302, is below the maximum pressure 304 for base flow operations, the controller 30 may be configured to increase the speed of the inflow pump 60 at a slower rate than was previously used. This may help limit the occurrence and/or magnitude of pressure overshoots.

It is further contemplated that the controller 30 may be configured to generate an audio and/or visual alert when the area under the curve 310 exceeds the predetermined area under the curve value. The alert may be an alphanumeric alert displayed at the touchscreen interface 42, a light (blinking or otherwise), a beep (or other sound), a haptic or vibrational alert, or the like. It is further contemplated that the controller 30 may be configured to display the area under the curve 310 results to the user at, for example, the touchscreen interface 42. In some cases, the controller 30 may be configured to display the calculated area under the curve 310 and/or the predetermined area under the curve value for an event regardless of whether the calculated area under the curve 310 exceeds the predetermined area under the curve value.

It is contemplated that the predetermined area under the curve value may be determined using data obtained from ureteroscopy procedures that result in pressurization of the kidney and urinary tract. In some cases, the predetermined area under the curve value may be determined using benchtop testing. In the following examples, pressure is measured in millimeters of mercury (mmHg) and time is measured in seconds(s). However, the pressure may be measured with other units, as desired and the present disclosure is not limited to mmHg. In one illustrative example, the predetermined area under the curve value for base flow operations may be in the range of about 130-150 mmHg·seconds for a single ILP overshoot. In another illustrative example, the predetermined area under the curve value for flush mode may be in the range of about 250-280 mmHg·seconds. The predetermined area under the curve value for either or both the base operations mode and flush mode may take into consideration the ILP will not immediately fall below the respective maximum pressure 304, 306 once predetermined area under the curve value is reached. The fluid management system 10 flush enables the physician to control a temporary increase in irrigation flow by holding down an appropriate button on the endoscope handle 218. While this button is held, the maximum pressure 306 for flush mode (e.g., the flush ILP limiter) is active, and overrides the maximum pressure 304 for base flow operations. To allow for a responsive flush feature, the fluid management system 10 may implement a “boost” phase, where the inflow pump 60 speeds up temporarily when flush is initiated to quickly pressurize the fluid cassette 110. The “boost” can lead to larger overshoots above the maximum pressure, and thus to enable a responsive design, a higher tolerance (e.g., higher area under the curve) on the flush ILP limiter may be required.

The controller 30 may continue to monitor the ILP 302 for excursions above the maximum pressure 304 for base operations or above the maximum pressure 306 for flush mode when flush is activated. During the procedure, the ILP 302 may exceed the maximum pressure 304, 306 more than once. In FIG. 4, the ILP 302 exceeds the maximum pressure 304 for base operations at a fourth time 318. The controller 30 may once again begin calculating the area under the curve 320. The area under the curve 320 may be incrementally summed with each pressure reading received from the pressure sensor 208 on the endoscope 20 until the ILP 302 drops or falls below the maximum pressure 304 for base operations at a later fifth time 322. If the area under the curve 320 does not exceed the predetermined area under the curve value for base operations, the controller 30 may not change a speed of the inflow pump 60, a direction of the inflow pump 60, and/or a speed of the outflow pump 33.

In FIG. 4, the ILP 302 exceeds the maximum pressure 304 for base operations at a sixth time 324. The controller 30 may once again begin calculating the area under the curve 326. The area under the curve 326 may be incrementally summed with each pressure reading received from the pressure sensor 208 on the endoscope 20 until the ILP 302 drops or falls below the maximum pressure 304 for base operations at a later seventh time 328. If the area under the curve 320 does not exceed the predetermined area under the curve value for base operations, the controller 30 may not change a speed of the inflow pump 60, a direction of the inflow pump 60, and/or a speed of the outflow pump 33.

The controller 30 may be further configured to calculate a running total of all pressure events (e.g., all the areas under the curve 310, 320, 326) that are recorded during the procedure. The controller 30 may be configured to compare the cumulative area under the curve 310, 320, 320 for the procedure to a predetermined cumulative area under the curve. This may be given as the integral equation:

$$AUC = {\int }_{time measured ILP exceeds reference}^{t} ( ILP - reference ) dt \qquad Equation 2$$

where AUC equals the area under the curve, ILP is the measure intraluminal pressure, and reference is the maximum ILP set by the user, a baseline pressure of the patient, or atmospheric pressure. The maximum ILP may vary depending on the mode of operation of the fluid management system 10. For example, as noted above, when the fluid management system 10 is in a flush mode, the maximum ILP may be greater than when the fluid management system 10 is in base flow mode.

If at any time cumulative area under the curve 310, 320, 326 exceeds the predetermined cumulative area under the curve, the controller 30 may be configured to control a speed of the inflow pump 60, a direction of the inflow pump 60 (e.g., the pump 60 may be reversed to draw fluid out), and/or a speed of the outflow pump 33 to lower the intraluminal pressure. In some cases, the controller 30 may be configured to lower the maximum pressure 304, 306 if the predetermined cumulative area under the curve has been met or exceeded. It is further contemplated that the controller 30 may be configured to generate an audio and/or visual alert when the cumulative area under the curve 310, 320, 326 exceeds the predetermined cumulative area under the curve. The alert may be an alphanumeric alert displayed at the touchscreen interface 42, a light (blinking or otherwise), a beep (or other sound), a haptic or vibrational alert, or the like.

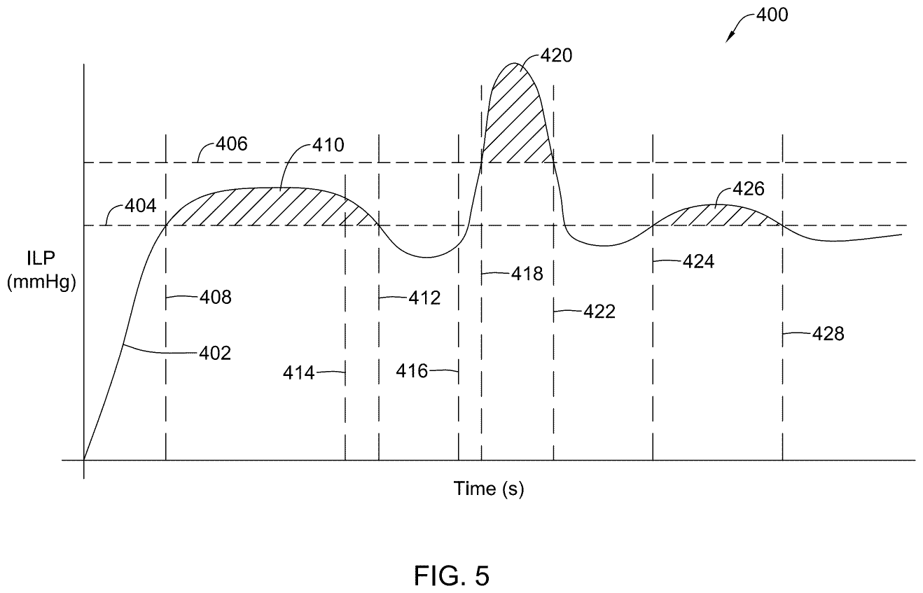

FIG. 5 is another illustrative graph 400 of intraluminal pressure 402 over time during a portion of a procedure. The intraluminal pressure 402 is transmitted from a pressure sensor 208 on the endoscope 200 to the controller 30. The fluid management system 10 may include flow limiter control settings which include a maximum ILP 404 for base flow operations or mode (or normal operation) and a maximum ILP 406 for flush mode or flush operation. As a greater volume of fluid may be introduced through the endoscope 200 for an expected shorter length of time the maximum ILP 406 for the flush mode may be greater than the maximum ILP 404 for flow mode. However, this is not required. The ILP may be measured in mmHg while time may be measured in seconds(s). The maximum ILP 404, 406 may be selected by the user at the touch screen interface 42 at the start of a procedure. For example, the user may select a maximum ILP 404, 406 between a range of about 5 mmHg to 400 mmHg. The maximum ILP 404 for base flow operations and the maximum ILP 406 for flush mode may be selected separately. However, the maximum ILP 406 for flush mode may be required to be at least equal to or greater than the maximum ILP 404 for base flow operations. When the flush mode is activated (e.g., via a button on the endoscope 200), the maximum ILP 406 for flush mode overrides the maximum ILP 404 for base flow operations.

At the beginning of a procedure, the endoscope 200 may be inserted into a body lumen of the patient. When the distal end 206 of the endoscope 200 is at or near the target location, a flow of fluid may be provided from the fluid management system 10 to the endoscope 200. As fluid is provided to the body lumen, the ILP 402 may gradually increase as fluid accumulates in the body lumen. In some cases, at some point during the procedure, the ILP 402 may exceed the maximum ILP 404. In the example illustrated in FIG. 5, the ILP 402 initially exceeds the maximum ILP 404 for flow operations at a first time 408. Once the ILP 402 exceeds the maximum ILP 404 for flow operations, the controller 30 may begin to integrate the ILP over time to determine the “area under the curve” illustrated as shaded region 410 between the measured ILP 402 and the maximum ILP 404 for flow operations. This may be given as Equation 1 above.

As the controller 30 is receiving the measured ILP 402 as discrete measurements at predetermined time intervals, the controller 30 may be configured to use numerical approximation to determine the area between each ILP measurement which are added together. Said differently, the controller 30 may use a trapezoidal approximation method. This may break the area under the curve 410 into small trapezoids using sequential pressure measurements and sum the incrementally obtained areas. It is contemplated that the controller 30 may be configured to cumulatively sum the area under the curve 410 as new pressure measurements are received. The controller 30 may be configured to continue to sum the area under the curve 410 until the ILP 402 drops below the maximum pressure 404 for base flow operations, as shown at time 412. Further, the controller 30 may be configured to compare each summation (e.g., the summation after each new pressure measurement is received) to a predetermined area under the curve value for an event. An event may be a single excursion above the maximum pressure 404 for base operations (or maximum pressure 406 for flush mode) from the time ILP 402 first exceeds the maximum pressure to the time the ILP 402 drops below the maximum pressure. For example, FIG. 5 illustrates three pressure events. The predetermined area under the curve value for a pressure event may be stored in a memory of the controller 30. While the calculation of the area under the curve is described with respect to the ILP 402 exceeding the maximum pressure 404 for base operations, the controller 30 is also configured to calculate the area under the curve when flush mode is active, using the maximum pressure 406 for flush mode as the trigger to start and stop calculating the area under the curve.

If at any time between the time 408 the ILP 402 exceeds the maximum pressure 404 for base flow operations and the time 412 the ILP 402 drops below the maximum pressure 404 for base flow operations the area under the curve 410 exceeds the predetermined area under the curve value (having units of pressure multiplied by time (e.g., mmHg·seconds)), the controller 30 may be configured to control a speed of the inflow pump 60, a direction of the inflow pump 60 (e.g., the pump 60 may be reversed to draw fluid out), and/or a speed of the outflow pump 33 to lower the intraluminal pressure. For example, the area under the curve 410 may exceed the predetermined area under the curve value at an intermediate time 414 between the first time 408 and the second time 412. The controller 30 may then transmit a control signal to the inflow pump 60 to reduce the pump speed to deliver less fluid to lower the ILP 402 without user intervention. The ILP 402 may continue to rise for a short period of time after the controller 30 reduces the pump speed. This may be due to compliance (e.g., flexibility) in the components of the fluid management system 10, the patient, etc. However, in some cases, the ILP 402 may not continue to rise. Once the ILP 402 is less than the maximum pressure 404 for base flow operations, the controller 30 may stop calculating the area under curve. In some examples, the controller 30 may store the area under curve 410 to use for analysis (e.g., future modifications of acceptable area under the curve values) and/or to determine a cumulative area under the curve for an entire procedure. Additionally, once the ILP 402, is below the maximum pressure 404 for base flow operations, the controller 30 may be configured to increase the speed of the inflow pump 60 at a slower rate than was previously used. This may help limit the occurrence and/or magnitude of pressure overshoots.

It is further contemplated that the controller 30 may be configured to generate an audio and/or visual alert when the area under the curve 410 exceeds the predetermined area under the curve value. The alert may be an alphanumeric alert displayed at the touchscreen interface 42, a light (blinking or otherwise), a beep (or other sound), a haptic or vibrational alert, or the like.

It is contemplated that the predetermined area under the curve value may be determined using data obtained from ureteroscopy procedures that result in pressurization of the kidney and urinary tract. In some cases, the predetermined area under the curve value may be determined using benchtop testing. In the following examples, pressure is measured in millimeters of mercury (mmHg) and time is measured in seconds(s). However, the pressure may be measured with other units, as desired and the present disclosure is not limited to mmHg. In one illustrative example, the predetermined area under the curve value for base flow operations may be in the range of about 130-150 mmHg·seconds for a single ILP overshoot. In another illustrative example, the predetermined area under the curve value for flush mode may be in the range of about 250-280 mmHg·seconds. The predetermined area under the curve value for either or both the base operations mode and flush mode may take into consideration the ILP will not immediately fall below the respective maximum pressure 404, 406 once predetermined area under the curve value is reached. The fluid management system 10 flush enables the physician to control a temporary increase in irrigation flow by holding down an appropriate button on the endoscope handle 218. While this button is held, the maximum pressure 406 for flush mode (e.g., the flush ILP limiter) is active, and overrides the maximum pressure 404 for base flow operations. To allow for a responsive flush feature, the fluid management system 10 may implement a “boost” phase, where the inflow pump 60 speeds up temporarily when flush is initiated to quickly pressurize the fluid cassette 110. The “boost” can lead to larger overshoots above the maximum pressure, and thus to enable a responsive design, a higher tolerance (e.g., higher area under the curve) on the flush ILP limiter may be required.

The controller 30 may continue to monitor the ILP 402 for excursions above the maximum pressure 404 for base operations or above the maximum pressure 406 for flush mode when flush is activated. During the procedure, the ILP 402 may exceed the maximum pressure 404, 406 more than once. In FIG. 5, the flush mode is activated at a fourth time 416. The ILP 402 increases in response to the increase in fluid flow. ILP 402 exceeds the maximum pressure 406 for flush mode operations at a fifth time 418. The controller 30 may once again begin calculating the area under the curve 420. The area under the curve 420 may be incrementally summed with each pressure reading received from the pressure sensor 208 on the endoscope 20 until the ILP 402 drops or falls below the maximum pressure 406 for flush mode at a later sixth time 422. If the area under the curve 420 does not exceed the predetermined area under the curve value for flush mode, the controller 30 may not change a speed of the inflow pump 60, a direction of the inflow pump 60, and/or a speed of the outflow pump 33. If the area under curve 420 exceeds the predetermined area under the curve value for flush mode at any time during the pressure event, the controller 30 may be configured to change a speed of the inflow pump 60, a direction of the inflow pump 60, and/or a speed of the outflow pump 33 in a similar manner to that described herein.

In FIG. 5, the ILP 402 exceeds the maximum pressure 404 for base operations at a seventh time 424. The controller 30 may once again begin calculating the area under the curve 426. The area under the curve 426 may be incrementally summed with each pressure reading received from the pressure sensor 208 on the endoscope 20 until the ILP 402 drops or falls below the maximum pressure 404 for base operations at a later eighth time 428. If the area under the curve 420 does not exceed the predetermined area under the curve value for base operations, the controller 30 may not change a speed of the inflow pump 60, a direction of the inflow pump 60, and/or a speed of the outflow pump 33.

The controller 30 may be further configured to calculate a running total of all pressure events (e.g., all the areas under the curve 410, 420, 426) that are recorded during the procedure. The controller 30 may be configured to compare the cumulative area under the curve 410, 420, 420 for the procedure to a predetermined cumulative area under the curve. This may be given as Equation 2 above.

If at any time cumulative area under the curve 410, 420, 426 exceeds the predetermined cumulative area under the curve, the controller 30 may be configured to control a speed of the inflow pump 60, a direction of the inflow pump 60 (e.g., the pump 60 may be reversed to draw fluid out), and/or a speed of the outflow pump 33 to lower the intraluminal pressure. In some cases, the controller 30 may be configured to lower the maximum pressure 404, 406 if the predetermined cumulative area under the curve has been met or exceeded. It is further contemplated that the controller 30 may be configured to generate an audio and/or visual alert when the cumulative area under the curve 410, 420, 426 exceeds the predetermined cumulative area under the curve. The alert may be an alphanumeric alert displayed at the touchscreen interface 42, a light (blinking or otherwise), a beep (or other sound), a haptic or vibrational alert, or the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for monitoring intraluminal pressure during endoscopic procedures, the system comprising:
a fluid management system configured to deliver fluid to a patient anatomy;
an endoscope configured to measure intraluminal pressure;
a controller configured to:
calculate an area under the curve metric between a maximum pressure and a measured intraluminal pressure over time when the measured intraluminal pressure exceeds the maximum value;
compare the calculated area under the curve metric to a predetermined area under the curve value; and
if the calculated area under the curve metric is greater than the predetermined area under the curve value, reduce a flow of fluid from the fluid management system.

2. The system of claim 1, wherein calculating the area under the curve metric comprises:
integrating the difference between the measured intraluminal pressure and the maximum pressure from a time when the intraluminal pressure exceeds the maximum pressure to a time when the intraluminal pressure returns below the maximum pressure.

3. The system of claim 1, wherein calculating the area under the curve metric comprises:
sampling the intraluminal pressure values at discrete time points; and
performing a numerical approximation using the sampled intraluminal values.

4. The system of claim 1, wherein the maximum pressure is determined by a mode of operation of the endoscope.

5. The system of claim 4, wherein a maximum pressure for a flush mode is greater than a maximum pressure for a base flow operation.

6. The system of claim 1, wherein if the calculated area under the curve metric exceeds the predetermined area under the curve value, the controller is configured to reduce a speed of an inflow pump delivering the fluid.

7. The system of claim 1, wherein the predetermined area under the curve value is between 130-150 mmHg·seconds for base flow operations and between 250-280 mmHg·seconds for flush operations.

8. The system of claim 1, wherein the controller is further configured to:
calculate a cumulative area under the curve metric for multiple pressure overshoots during a procedure; and
compare the cumulative area under the curve metric to a predetermined cumulative value.

9. The system of claim 1, wherein the controller is configured to generate an alert when the calculated area under the curve metric exceeds the predetermined area under the curve value.

10. The system of claim 1, wherein the controller is configured to increase fluid flow at a reduced rate after the measured intraluminal pressure returns below the maximum pressure following an overshoot event.

11. A non-transitory computer-readable medium storing instructions that, when executed by a processor, cause the processor to:
receive intraluminal pressure measurements from a pressure sensor during a procedure;
detect when a measured intraluminal pressure exceeds a maximum pressure value;
calculate an area under the curve metric between the maximum pressure value and the measured intraluminal pressure over time when the measured intraluminal pressure exceeds the maximum value;
compare the calculated area under the curve metric to a predetermined area under the curve value; and
if the calculated area under the curve metric exceeds the predetermined area under the curve value, generate a control signal to reduce fluid flow.

12. The non-transitory computer-readable medium of claim 11, wherein calculating the area under the curve metric comprises:
sampling the intraluminal pressure measurements at discrete time points; and
performing a trapezoidal numerical approximation using the intraluminal pressure measurements.

13. The non-transitory computer-readable medium of claim 11, wherein the predetermined area under the curve value is between 130-150 mmHg·seconds for base flow operations and between 250-280 mmHg·seconds for flush operations.

14. The non-transitory computer-readable medium of claim 11, wherein the instructions further cause the processor to:
calculate a cumulative area under the curve metric for multiple pressure overshoots during the procedure; and
compare the cumulative area under the curve metric to a predetermined cumulative specification.

15. The non-transitory computer-readable medium of claim 1, wherein the instructions further cause the processor to:
generate an alert when the calculated area under the curve metric exceeds the predetermined area under the curve value.

16. A method of evaluating intraluminal pressure safety during a procedure, comprising:
receiving intraluminal pressure measurements from a pressure sensor during fluid delivery to a patient anatomy;
detecting when measured intraluminal pressure exceeds a maximum pressure value;
calculating an area under the curve metric between the maximum pressure value and the measured intraluminal pressure over time when the measured intraluminal pressure exceeds the maximum value;
comparing the calculated area under the curve metric to a predetermined area under the curve value; and
if the calculated area under the curve metric exceeds the predetermined area under the curve value, reducing a flow rate of fluid delivery.

17. The method of claim 16, wherein calculating the area under the curve metric comprises:
sampling the intraluminal pressure measurements at discrete time points; and
performing a trapezoidal numerical approximation using the intraluminal pressure measurements.

18. The method of claim 16, wherein the predetermined area under the curve value is between 130-150 mmHg·seconds for base flow operations and between 250-280 mmHg·seconds for flush operations.

19. The method of claim 16, further comprising:
calculating a cumulative area under the curve metric for multiple pressure overshoots during the procedure; and
comparing the cumulative area under the curve metric to a predetermined cumulative specification.

20. The method of claim 16, further comprising:
after reducing the flow rate of fluid delivery, increasing the flow rate at a reduced rate when the measured intraluminal pressure returns below the maximum pressure value.
